(19) United States
(12) Patent Application Publication
(10) Pub. No.: US 2010/0015079 A1
(43) Pub. Date: Jan. 21, 2010

(54) Cosmetic Compositions for the Protection or Regeneration of Hair or Skin

(75) Inventor: Karl-Heinz Schrader, Bevern (DE)

Correspondence Address: VIKSNINS HARRIS & PADYS PLLP, P.O. BOX 111098, ST. PAUL, MN, 55111-1098, US

(73) Assignee: Institut Dr. Schrader

(21) Appl. No.: 12/298,005

(22) PCT Filed: Apr. 19, 2007
(86) PCT No.: PCT/EP2007/053863

§ 371 Date: Apr. 9, 2009

(30) Foreign Application Priority Data
Apr. 21, 2006 (EP) 06008335.9

(51) Int. Cl. A61K 8/46; A61Q 5/00; A61Q 19/00

(52) U.S. Cl. 424/70.31; 514/601; 514/603; 514/517

(57) ABSTRACT

The present invention relates to a cosmetic composition which comprises a compound of the formula R²R³N—(CH₂)ₙ—SO₂R¹, in which R¹ is OR⁴ or NR⁵R⁶; R² and R³, independently of one another, are H, C₁-C₆-alkyl, phenyl-C₁-C₄-alkyl, HCO or C₁-C₄-alkyl-CO; R⁴ is C₁-C₆-alkyl; R⁵ and R⁶, independently of one another, are H or C₁-C₆-alkyl; and n is 2, 3 or 4.

The cosmetic compositions can be used for protecting the hair and the skin against oxidative damage and for regenerating oxidatively damaged hair or oxidatively damaged skin.

The present invention relates to cosmetic compositions for protecting the hair or the skin against oxidative damage and for regenerating oxidatively damaged hair or oxidatively damaged skin.

An essential constituent of hair and of skin is keratin, which plays a decisive role in determining the properties of the hair. The keratin is often damaged by external influences. For example, solar irradiation (irradiation with UV light) leads to a reduction in the mechanical strength of the hair and to a bleaching of the natural and artificial hair color. Chlorinated water in swimming baths, pools etc. causes oxidation of the keratin, which can lead to a loss of protein. Moreover, the hair is often subjected to chemical hair treatments, such as treatments with permanent waving or hair straightening compositions, hair colorants, bleaches, shampoos, etc. Just like UVA irradiation, these treatments can result in the formation of hydrogen peroxide, which can lead to interactions and/or color-changing effects on the hair. Bleach stresses generally lead to hair damage, where the melanine pigments in the cuticle cells and the cortex cells of the hair are degraded by complex chemical reactions. This results in changes to the original properties of the hair. Damaged hair is more rough, more brittle and more difficult to comb. It is more porous and therefore more sensitive to the effect of moisture and therefore requires longer drying times. Its resistance and tear strength are reduced and its extensibility is altered. Since the noxae involved in the hair treatment cannot be eliminated without residue by rinsing, the result is ultimately insidious oxidation and consequently the specified hair damage associated with color changes.

There is a large number of approaches for limiting such damage. Antioxidative agents are often used for this purpose, especially those which are able to degrade hydrogen peroxide and scavenge active oxygen and free radicals. One such agent is described in U.S. Pat. No. 5,601,806, in which an aminothiosulfonic acid is used as antioxidant.

It is common to the prior art that the effect of the agents known hitherto is unsatisfactory or the agents are incompatible with cosmetic agents.

The object of the present invention is therefore to provide a cosmetic composition which is able to effectively protect the hair and the skin against oxidative damage and/or to reduce the damage to the skin.

Surprisingly, it has now been found that this object is achieved by a cosmetic composition which comprises a taurine derivative.

The present invention therefore provides a cosmetic composition comprising a compound of formula I

R²R³N—(CH₂)ₙ—SO₂R¹ (I)

in which R¹ is OR⁴ or NR⁵R⁶;
R² and R³, independently of one another, are H, C₁-C₆-alkyl, phenyl-C₁-C₄-alkyl, HCO or C₁-C₄-alkyl-CO;
R⁴ is C₁-C₆-alkyl;
R⁵ and R⁶, independently of one another, are H or C₁-C₆-alkyl; and
n is 2, 3 or 4,
or a salt thereof.

Preferably, R¹ is NR⁵R⁶, where R⁵ and R⁶ have the meanings given above and are in particular H.

R² and R³ are preferably H or C₁-C₆-alkyl and in particular H.

n is in particular 2.

A particularly preferred embodiment is a cosmetic composition which comprises taurinamide of the formula

H₂N—(CH₂)₂—SO₂NH₂

or a salt thereof.

Suitable salts of the compounds of formula I are salts with inorganic or organic acids. Inorganic acids which can be used are, for example, hydrochloric acid, hydrobromic acid, sulfuric acid or phosphoric acid. Organic acids which can be used are, for example, acetic acid, propionic acid, lactic acid, malic acid, adipic acid, aspartic acid, citric acid, mandelic acid, fumaric acid, maleic acid, succinic acid, tartaric acid, methanesulfonic acid or toluenesulfonic acid.

The compounds of formula I in which R¹ is NR⁵R⁶ can be prepared by methods described in WO 96/34854 and EP 863 133. The starting material is a compound of the formula

R^{a}CH₂CH₂SO₂R^{b}

in which R^{a} and R^{b} are a halogen atom, R^{b} may also be an azide radical or R^{a} and R^{b} together are —O—SO₂—O—. These compounds are reacted with an azide compound, for example an alkali metal azide, giving P-azidoethanesulfonylazide, which can, for example, be reduced to taurinamide by catalytic hydrogenation. The alkyl or acyl groups bonded to the nitrogen atoms are introduced in the usual manner through reaction with an alkylating agent, such as an alkyl halide, or an acylating agent, such as a carboxylic acid anhydride or carboxylic acid chloride.

The compounds of formula I in which R¹ is OR⁴ can be prepared starting from the compounds described in EP 863 133 A2 of the formula

HX—H₂NCH₂CH₂SO₂X′

in which X and X′ are halogen, in particular Cl or Br, through reaction with the alcohol R⁴OH and, if appropriate, introduction of the alkyl or acyl groups on the nitrogen atom (see above).

The preparation of those compounds of formula I in which n is 3 or 4 can take place in an analogous manner.

The compositions according to the invention can be formulated as skin treatment compositions or hair treatment compositions and comprise all carriers, active ingredients and auxiliaries customarily used in these fields. The skin treatment compositions are in particular in the form of an emulsion, gel, lotion, oil or solution. The hair treatment compositions can be formulated as shampoo, conditioner, gel, cream, lotion, spray, tincture or solution. The compositions according to the invention preferably have a pH in the range from 2 to 10, in particular 3.5 to 10 and particularly preferably 3.5 to 8.

Suitable carriers and auxiliaries are, for example:

solvents, such as water, water-miscible alcohols, e.g. ethanol or isopropanol and mixtures thereof;
oils, such as soybean oil, peanut oil, olive oil, sunflower oil, sesame oil, avocado oil, lauryl alcohol, myristyl alcohol, cetyl alcohol, stearyl alcohol, myristic acid, stearic acid, palmitic acid, oleic acid, etc.;
surfactants, such as
anionic surfactants, for example alkyl sulfates, alkyl polyglycol ether sulfates and ether carboxylic acids having 10 to 18 carbon atoms in the alkyl group and up to 12 glycol ether groups in the molecule;
nonionic surfactants, such as ethoxylated fatty alcohols having 8 to 30 carbon atoms, ethoxylated fatty acids having 8 to 30 carbon atoms and ethoxylated alkylphenols having 8 to 15 carbon atoms in the alkyl group;
amphoteric surfactants, alkylamidopropylbetaines (Tego® betaine series), amine oxides or imidazolinium betaines (Miranol® series);
cationic surfactants, such as quaternary ammonium compounds, for example alkyltrimethylammonium chlorides, such as cetyltrimethylammonium chloride, stearyltrimethylammonium chloride etc., compositions which comprise at least one ester function and at least one quaternary ammonium group, for example ester salts of fatty acids with triethanolamine, alkylamidoamines etc.

Emulsifiers of the O/W or W/O type;

cationic polymers, as are listed, for example, in WO 02/74265, to which reference is made here in its entirety. Preferred cationic polymers are those of the polyquaternium series (see Fiedler, Encyclopedia of Excipients, 5^{th} edition, 2002, pages 1273-1276), quaternized cellulose derivatives, such as the commercial products Celquat® and Polymer JR®, cationic guar derivatives, polysiloxanes with quaternary groups, polymeric dimethyldiallylammonium salts (Merquat® series, see Fiedler, Encyclopedia of Excipients, 2002, pages 1023 and 1024), copolymers of vinylpyrrolidone with quaternized derivatives of dialkylaminoalkyl acrylate and methacrylate, which are available under the trade name Gafquat® etc.;
nonionic polymers, such as vinylpyrrolidone/vinyl ester copolymers, cellulose ethers, polyvinylpyrrolidones, siloxanes etc.;
protein hydrolyzates;
vitamins;
thickeners, such as guar gum, alginates, xanthan gum, gum Arabic, cellulose derivatives etc.

As regards further components of the compositions according to the invention, reference is made to the relevant handbooks, for example the monograph by K. H. Schrader, Grundlagen und Rezepturen der Kosmetika [Fundamentals and formulations of cosmetics], 2^{nd} edition, Hüthig Buchverlag Heidelberg, 1989.

The hair treatment compositions according to the invention can also be formulated as colorants, permanent waving compositions or compositions for straightening the hair and which comprise auxiliaries required therefor.

The compounds of the formula I are present in the compositions according to the invention generally in an amount of from 0.1% by weight to 8% by weight, in particular 0.5% by weight to 5% by weight and particularly preferably 0.5% by weight to 3% by weight, in each case based on the total weight of the composition. The compositions according to the invention are applied to the hair or the skin in the usual manner. If the compositions are in the form of a shampoo or conditioner, they are generally rinsed out with water. If they are in the form of a conditioner, treatment cream, gel or foam, they may also remain on the hair without being rinsed out. Moreover, the compositions can be formulated in two or more parts, as is often the case particularly with permanent wave compositions and colorants.

The compositions according to the invention are able to suppress or to reduce the formation of active oxygen species (superoxides, hydrogen peroxide, hydroxyl radicals, singlet oxygen, hyperchlorous acid) and of free radicals (lipid peroxide free radicals, lipid alkoxy free radicals, lipid free radicals), which are formed primarily as a result of irradiation with visible light and UV light or as a result of hair treatment with oxidizing agents. They can therefore be used for protecting the hair and the skin against oxidative damage or for regenerating oxidatively damaged hair or oxidatively damaged skin. Using the compositions according to the invention, it is possible to avoid or reduce color-changing damage to the hair and the skin. Moreover, the damage to the structure of the hair and the skin is avoided or reduced such that the natural properties of hair and skin are retained or are at least partially restored. In particular, the resistance, tear strength, extensibility and combability are retained or, in the case of hair which is already damaged, are significantly improved.

The invention therefore also provides the use of a compound of formula I

R²R³N—(CH₂)ₙ—SO₂R¹ (I)

in which R¹ is OR⁴ or NR⁵R⁶;
R² and R³, independently of one another, are H, C₁-C₆-alkyl, phenyl-C₁-C₄-alkyl, HCO or C₁-C₄-alkyl-CO;
R⁴ is H or C₁-C₆-alkyl;
R⁵ and R⁶, independently of one another, are H or C₁-C₆-alkyl; and
n is 2, 3 or 4,
or a salt thereof for protecting the hair and the skin against oxidative damage or for regenerating oxidatively damaged hair or oxidatively damaged skin or as conditioner for hair and skin.

For the use according to the invention, the compounds are used as cosmetic compositions as described above.

The examples below illustrate the invention without limiting it.

### EXAMPLE 1

The effect of taurinamide hydrochloride on the regeneration of permanently waved hair was investigated. For this, three tresses of European hair (left natural, dense, color 7.0; Kerling) were subjected to a permanent wave treatment. The permanent wave solution had the following composition:

| | | |
|---|---|---|
| Demineralized water | 74.79% | by wt. |
| Sodium carbonate | 7.14% | by wt. |
| Thioglycolic acid | 9% | by wt. |
| Ammonia solution, 25% strength | 9% | by wt. |
| Texapon K12P (sodium lauryl sulfate) | 0.07% | by wt. |

| | |
|---|---|
| pH | 9.6 |

The neutralizing solution had the following composition:

| | | |
|---|---|---|
| Demineralized water | 93.7% | by wt. |
| Hydrogen peroxide, 30% strength | 5.7% | by wt. |
| Turpinal SL (etidronic acid) | 0.1% | by wt. |
| Texapon K12P (sodium lauryl sulfate) | 0.5% | by wt. |

| | |
|---|---|
| pH | 2.6 |

The tresses were washed for 1 min in a shaking machine (13.5% Texapon K12P in demineralized water) and rinsed for 2 minutes with lukewarm water. The tresses were saturated with permanent waving solution until drop-wet (10 ml for 3 tresses). After a contact time of 30 minutes, they were rinsed for 3 minutes with lukewarm water. The treatment with the permanent waving solution was repeated three times. The drop-wet tresses were then saturated with neutralizing solution (10 ml for 3 tresses). After a contact time of 10 minutes, they were rinsed for 3 minutes with lukewarm water.

To determine the effect of taurinamide, the following rinse-off formulations which comprised varying amounts of taurinamide hydrochloride were used:

Formulation

| Components (% by wt.) | 1.1 | 1.2 | 1.3 | 1.4 |
|---|---|---|---|---|
| Demineralized water | 91.4 | 91 | 90.5 | 86.5 |
| Lanette 16 (cetyl alcohol) | 8 | 8 | 8 | 8 |
| Texapon K12P | 0.5 | 0.5 | 0.5 | 0.5 |
| (sodium lauryl sulfate) | | | | |
| Taurinamide•HCl | 0.1 | 0.5 | 1 | 5 |
| NaOH, 10% strength to pH | 7.2 | 6.9 | 6.9 | 6.9 |

The formulations were applied to the tresses from both sides using a paint brush on a glass plate (30 g for 3 tresses). After a contact time of 10 minutes, they were rinsed for 3 minutes with lukewarm water. The hair was then left to dry in the air.

Two days after the treatment, the UVA-induced photon emission (chemiluminescence) was measured. The photon emission is a measure of the hair damage. It was measured in accordance with the method described by S. Benard, H. Nerenz, M. Rohr and A. Schrader in IFSCC magazine Vol. 4 (2001), 185-189 (ICL-H (Induced Chemiluminescence of Human Hair) A new method for quantitative analysis of hair stress). The following results were obtained:

| | |
|---|---|
| Damage by permanent wave | 555% |
| 0.1% Taurinamide | 329% |
| 0.5% Taurinamide | 209% |
| 1% Taurinamide | 185% |
| 5% Taurinamide | 118% |

It is clear that a treatment with taurinamide significantly reduces hair damage.

### EXAMPLE 2

The experiments described in Example 1 were repeated but using 0.1% by weight or 0.5% by weight of taurine hydrochloride instead of taurinamide hydrochloride. The results were as follows:

| | |
|---|---|
| Damage by permanent wave | 471% |
| after treatment with | |
| 0.1% Taurine | 350% |
| 0.5% Taurine | 324% |

1. A cosmetic composition comprising a compound of formula I
R²R³N—(CH₂)ₙ—SO₂R¹
in which R¹ is OR⁴ or NR⁵R⁶;
R¹ and R³, independently of one another, are H, C₁-C₆-alkyl, phenyl-C₁-C₄-alkyl, HCO or C₁-C₄-alkyl-CO;
R⁴ is C₁-C₆-alkyl;
R⁵ and R⁶, independently of one another, are H or C₁-C₆-alkyl; and
n is 2, 3 or 4,
or a salt thereof.

2. The composition as claimed in claim 1, where R¹ is NR⁵R⁶.

3. The composition as claimed in claim 2, where R⁵ and R⁶ are H.

4. The composition as claimed in claim 1, where R² and R³, independently of one another, are H or C₁-C₆-alkyl.

5. The composition as claimed in claim 1, where n is 2.

6. The composition as claimed in claim 1 in the form of a hair treatment composition.

7. A method of protecting hair and skin against oxidative damage or of regenerating oxidatively damaged hair or oxidatively damaged skin, which comprises applying a compound of formula I
R²R³N—(CH₂)ₙ—SO₂R¹ (I)
in which R¹ is OR⁴ or NR⁵R⁶;
R² and R³, independently of one another, are H, C₁-C₆-alkyl, phenyl-C₁-C₄-alkyl, HCO or C₁-C₄-alkyl-CO;
R⁴ is H or C₁-C₆-alkyl;
R⁵ and R⁶, independently of one another, are H or C₁-C₆-alkyl; and
n is 2, 3 or 4,
or a salt thereof to the hair or skin.

8. The method as claimed in claim 7, where R¹ is OH or NR⁵R⁶.

9. The method as claimed in claim 7, where R¹ is NR⁵R⁶ and R⁵ and R⁶ are H.

10. The method as claimed in claim 7, where R² and R³, independently of one another, are H or C₁-C₆-alkyl.

11. The method as claimed in claim 7, where n is 2.

12. The method as claimed in claim 7, where the compound of formula I is applied in the form of a conditioner for hair and skin.
